(19) United States
(12) Patent Application Publication
(10) Pub. No.: US 2023/0270681 A1
(43) Pub. Date: Aug. 31, 2023

(54) DRUG DELIVERY AGENTS FOR PREVENTION OR TREATMENT OF PULMONARY DISEASE

(71) Applicant: UNIVERSITY INDUSTRY FOUNDATION YONSEI UNIVERSITY WONJU CAMPUS, Wonju-si (KR)

(72) Inventors: Jaehong KEY, Wonju-si (KR); Sanghyo PARK, Wonju-si (KR)

(21) Appl. No.: 18/114,168

(22) Filed: Feb. 24, 2023

Related U.S. Application Data
(63) Continuation-in-part of application No. 17/108,403, filed on Dec. 1, 2020, now abandoned.
Application No. 17/108,403 is a continuation-in-part of application No. PCT/KR19/06332, filed on May 27, 2019.

(30) Foreign Application Priority Data
Jun. 1, 2018 (KR) 10-2018-0063486

(51) Int. Cl. A61K 9/20; A61K 31/496; A61K 51/12; A61K 49/00; A61P 11/00; A61P 35/00; A61K 31/704; A61P 31/12

(52) U.S. Cl. CPC A61K 9/204; A61K 31/496; A61K 51/1244; A61K 49/0091; A61P 11/00; A61P 35/00; A61K 31/704; A61P 31/12

(57) ABSTRACT

Provided is a lung disease drug delivery carrier. The lung disease drug delivery carrier includes a disc particle having a diameter of 2 μm to 4 μm. The disc particle is injected into the human body. The disc particle includes a polymer selected from the group consisting of polyglycolic acid (PGA), polylactide (PLA), polyglycolide (PG), polyphosphazene, polyiminocarbonate, polyphosphoester, polyanhydride, polyorthoester, and combinations thereof, polylactide-co-glycolide (PLGA), and a drug. The disc particle is decomposed after 24 hours after being injected into the human body and delivers or releases the drug into a lung. The lung disease drug delivery carrier is accumulated in the lung, and the lung disease includes pulmonary fibrosis.

### BACKGROUND

### Field

The present disclosure relates to a lung disease drug delivery carrier that accumulates intensively in lung diseases such as lung cancer.

### Description of the Related Art

The lungs have a very large overall surface area. A thickness of the cells constituting the alveolar sac is 0.1 μm to 0.5 μm, and thus is very small. A density of the cells thereof is lower than that of other cells, so that drug absorption thereto is easy. When a drug is delivered through the lungs, the speed of the systemic circulation of the drug is fast and the drug is not subjected to the first pass metabolism. Thus, the delivery through the lung is very suitable as an administration route of immediate-release drug formulations and has been known as an effective route for local diseases such as asthma/chronic bronchial obstruction.

Further, due to the above characteristics, lung cells exhibit high membrane permeability to macromolecules, and the amount of bioenzymes present in the lung mucosa is relatively small. Thus, the delivery of the drug through the lung is known as effective injection-dependent protein and peptide drug delivery route in the body. In fact, in various literatures, it has been reported that the maximum time to reach the maximum blood concentration of these drugs is about 30 minutes and the bioavailability of these drugs reaches 50% (Leuprolide) compared to the subcutaneous route. In addition, research on drug delivery systems and delivery media through the respiratory tract is actively being conducted due to the improvement of patient convenience that they may take medication on their own.

In the meantime, the drug delivery system refers to a generic term of a series of technologies that control the delivery and release of substances with pharmacological activity to cells, tissues, and organs to exert optimal effects using various physicochemical technologies. The drug delivery system refers to a technology that optimizes drug treatment by designing a formulation to minimize side effects of existing drugs, maximize efficacy and effect, and efficiently deliver the required amount of drug.

Drug delivery systems may be classified based on the route of administration, the type of delivery technology, and the type of drug. Classification based on route of administration may generally include oral, injection, pulmonary inhalation, transdermal, implantation, etc. The classification based on the type of delivery technology may include absorption promotion type, drug effect sustaining type, target site concentration type, and Intelligent DDS.

A carrier for delivering a drug in the drug delivery system as described above may include microparticles or microspheres. It is important to design a formulation to reduce the side effects of the drug, increase patient compliance with the drug, and maximize the efficacy and effect of the drug by efficiently delivering the drug for disease treatment to the treatment site using these drug carriers.

Particularly, the microparticles for drug delivery using biodegradable polymers should be able to easily contain fat-soluble or water-soluble bioactive substances in the microparticles. The microparticles for drug delivery using biodegradable polymers should have properties that the microparticle may contain drugs in the human body and maintain the drugs therein for a certain period of time, safety in decomposition thereof into substances harmless to the human body, and persistence that the microparticle does not release the drug at the initial stage of being injected into the human body, but must have to release the drug for the desired period after reaching the target point.

International Publication No. WO 2015/176025 discloses a non-spherical nano/fine particle and a preparation method thereof used for diagnosis of cancer treatment. In the above patent, a method of delivering a drug including a contrast agent and a therapeutic agent to cells and/or tissues of the body using non-spherical nano/fine particles is known.

### SUMMARY

A purpose of the present disclosure is to provide a lung disease drug delivery carrier that intensively accumulates in lung diseases such as lung cancer.

However, the technical purpose to be achieved by examples of the present disclosure may not be limited to the technical purposes as described above. Other technical challenges may be present.

As a technical means for addressing the above technical problem, a first aspect of the present disclosure provides a lung disease drug delivery carrier in which a drug is introduced into a disc particle including polylactide-co-glycolide (PLGA), wherein the carrier delivers and/or releases the drug therein into the lung, wherein the disc particle is 1 μm to 5 μm in size.

According to one implementation of the present disclosure, the disc particle may have a size of 3 μm, but may not be limited thereto.

According to one implementation of the present disclosure, the drug may include a drug selected from the group consisting of therapeutic agents, contrast agents, diagnostic agents, and combinations thereof, but may not be limited thereto.

According to one implementation of the present disclosure, the disc particle may be decomposed after 24 hours, but may not be limited thereto.

According to one implementation of the present disclosure, the disc particle may further include a polymer selected from the group consisting of polyglycolic acid (PGA), polylactide (PLA), polyglycolide (PG), polyphosphazene, polyiminocarbonate, polyphosphoester, polyanhydride, polyorthoester, and combinations thereof, but may not be limited thereto.

According to one implementation of the present disclosure, the therapeutic agent may include one selected from the group consisting of chemotherapeutic compounds, anti-inflammatory agents, anticancer agents, and combinations thereof, but may not be limited thereto.

According to one implementation of the present disclosure, the therapeutic agents may include, but may not be limited to, those selected from the group consisting of cytotoxic agents, cell arresters, alkylating agents, metabolic antagonists, anti-tumor antibiotics, DNA polymerase inhibitors, DNA gyrase inhibitors, topoisomerase inhibitors, mitosis inhibitors, corticosteroids, intercalating agents, antibodies, hormones, antagonists, and combinations thereof.

According to one implementation of the present disclosure, the chemotherapeutic compounds may include those selected from the group consisting of doxorubicin, vinblastine, vincristine, fludarabine, carmustine, asparaginase, fluorouracil, methotrexate, cyclophosphamide, carboplatin, bleomycin, daunorubicin, lomustine, irinotecan, paclitaxel, docetaxel, etoposide, gemcitabine, imatinib, flutamide, hydroxyurea, trastuzumab, curcumin, temozolomide, and combinations thereof but may not be limited thereto.

According to one implementation of the present disclosure, the drug may include an isotope for nuclear imaging or radiotherapy, but may not be limited thereto.

According to one implementation of the present disclosure, the isotope may include one selected from the group consisting of ⁸⁹Zr, ⁶⁴Cu, ⁶⁸Ga, ⁹⁰Y, ¹⁷⁷Lu, and combinations thereof, but may not be limited thereto.

According to one implementation of the present disclosure, the nuclear imaging may include positron emission tomography (PET), but may not be limited thereto.

According to one implementation of the present disclosure, the contrast agent may include one selected from the group consisting of USPIO, SPIO, Gd chelate, magnetic nanoparticles, and combinations thereof, but may not be limited thereto.

According to one implementation of the present disclosure, the contrast agent may include an optical activator, but may not be limited thereto.

According to one embodiment of the present disclosure, the optical activator may include a dye selected from the group consisting of fluorescent dyes, cyanine, coumarin, anthracene, acridine, Texas red, fluorescein isothiocyanate (FITC), and combinations thereof, but is not limited thereto.

According to one embodiment of the present disclosure, the optical activator may include a chromophore including a fluorescent chromophore, but is not limited thereto.

According to one embodiment of the present disclosure, the optical activator may include a fluorescent molecule selected from the group consisting of a green fluorescent protein, a fluorescent chromophore, fluorescein isothiocyanate (FITC), and combinations thereof, but is not limited thereto.

The above-described means of solving the problems are merely exemplary and should not be construed as limiting the present disclosure. In addition to the above-described examples, additional examples may be derived from the drawings and detailed description.

According to the above-described means of solving the problem of the present disclosure, the lung disease drug delivery carrier according to the present disclosure uses the biodegradable polymer to be decomposed in the body after 24 hours after injection thereto, and is harmless to human body.

The conventional drug carrier prepared in an emulsion manner has a small size and may have a low drug load amount of around 10 wt %. The time for the carrier to stay in the lungs is also short. However, the lung disease drug delivery carrier according to the present disclosure may have a high drug load amount of around 50 wt %. Due to the size and shape of the disc shape, the drug may be delivered effectively to the target site because the carrier stays longer in the lungs of patients with lung disease compared to normal lungs.

Further, when the drug is delivered directly, the amount of accumulation in the lungs as well as the surrounding organs is large. However, the lung disease drug delivery carrier according to the present disclosure is intensively accumulated in the lungs and a relatively small amount thereof is accumulated in the normal lung for a short period of time. The intensive diagnosis or treatment of lung disease may be achieved.

The effects according to the present disclosure are not limited to the contents exemplified above, and more various effects are included in the present specification.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a schematic diagram of a disc particle preparation process according to one Example of the present disclosure;

FIG. 2 is a scanning electron microscope image of a silicon mold according to one Example of the present disclosure;

(a) OF FIG. 3 is a scanning electron microscope image of a disc particle with a width of 1 μm; (b) OF FIG. 3 is a scanning electron microscope image of a disc particle containing cyanine according to one Example of the present disclosure; (c) OF FIG. 3 is a scanning electron microscope image of a 5 μm width disc particle;

FIG. 4 is a graph of a size distribution of disc particles according to one example of the present disclosure;

FIG. 5 is a graph of distribution of the number of disc particles according to one example of the present disclosure;

FIG. 6 is a graph of the absorbance of a drug of a lung disease drug delivery carrier according to one example of the present disclosure;

(a) OF FIG. 7 is a table of drug loading amounts of lung disease drug delivery carriers according to one Example of the present disclosure and Comparative Example; (b) OF FIG. 7 is a graph of the drug release amount of the lung disease drug delivery carrier according to one Example of the present disclosure;

FIG. 8 is an optical microscope image of the lung disease drug delivery carrier according to one Example of the present disclosure and doxorubicin;

FIG. 9 is an optical microscope image of major organs 3 hours after injecting a lung disease drug delivery carrier according to one Example of the present disclosure and Comparative Example into a lung cancer metastasis model;

FIG. 10 is an optical microscope image of a lung cancer metastasis model injected with a lung disease drug delivery carrier according to one Example of the present disclosure;

FIG. 11 is a computed tomography and positron emission tomography image of a lung cancer metastasis model injected with a lung disease drug delivery carrier according to one Example of the present disclosure; and

FIG. 12 is a positron emission tomography image of a lung cancer metastasis model and a normal model injected with a lung disease drug delivery carrier according to one Example of the present disclosure.

FIG. 13A is a graph showing the degree of decomposition according to a molar ratio of lactic acid in PLGA disc particles according to one embodiment of the present disclosure, and FIG. 13B is a graph showing the degree of decomposition according to a molar ratio of lactic acid in drug-loaded PLGA disc particles according to one embodiment of the present disclosure.

FIGS. 14A and 14B are graphs showing the degree of release of nintedanib over time from nintedanib-loaded PLGA disc particles according to one embodiment of the present disclosure.

FIG. 15A illustrates an effect of nintedanib-loaded PLGA disc particles according to one embodiment of the present disclosure, and FIGS. 15B to 15E illustrate experimental results according to each comparison group.

FIG. 16 is an SEM image expressing the shape of PLGA disc particles according to one embodiment of the present disclosure.

FIGS. 17A and 17B are micro-CT images of a control group, a BLM administered group, a BLM and Nib administered group, and a BLM and Nib-DPPs administered group.

FIG. 18A is an image of photographing the locations of cancer tumor and the locations of an injected DOX-DPP (doxorubicin-loaded disc-shaped drug delivery carrier) in an animal model, and FIG. 18B illustrates comparison of the locations of cancer tumor in major organs and amounts of doxorubicin alone (free DOX) or DOX-DPP in major organs.

FIG. 19A is a schematic diagram expressing a treatment time after injecting cancer cells into an animal (rat), and FIG. 19B illustrates lung specimens of C57BL/6 mice with lung cancer, which are treated with saline, a drug delivery carrier (DPP), doxorubicin (free DOX), and a doxorubicin-loaded drug delivery carrier (DOX-DPP). FIG. 19C is a graph showing a change in survival rate of rats according to treated drugs over time, FIG. 19D is a graph showing a change in body weight of rats according to treated drugs over time, and FIG. 19E illustrates lung tissues of rats according to treated drugs.

FIG. 20 illustrates tissue images of each organ of rats according to treated drugs.

FIGS. 21A and 21B are lung tissue images of rats with asthma according to treated drugs.

FIG. 22A is a diagram showing an in-vivo distribution of radioactive isotope-labeled disc-shaped drug delivery carriers, and FIG. 22B is an image of photographing rats injected with the disc-shaped drug delivery carrier.

### DETAILED DESCRIPTION OF THE EMBODIMENT

Hereinafter, an example of the present disclosure will be described in detail with reference to the accompanying drawings so that a person having ordinary knowledge in the technical field to which the present disclosure belongs may easily implement the same. However, the present disclosure may be implemented in many different forms and may not be limited to the example described herein. Further, in the drawings, in order to clearly explain the present disclosure, parts irrelevant to the description are omitted. Similar reference numerals are attached to similar parts throughout the specification.

It will be understood that when an element or layer is referred to as being “connected to”, or “coupled to” another element or layer, it may be directly on, connected to, or coupled to the other element or layer, or one or more intervening elements or layers may be present. In addition, it will also be understood that when an element or layer is referred to as being “between” two elements or layers, it may be the only element or layer between the two elements or layers, or one or more intervening elements or layers may also be present.

In addition, it will also be understood that when a first element or layer is referred to as being present “on” a second element or layer, the first element may be disposed directly on the second element or may be disposed indirectly on the second element with a third element or layer being disposed between the first and second elements or layers.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or more other features, integers, operations, elements, components, and/or portions thereof.

The terms “about”, “substantially”, etc. in the present disclosure are used to indicate inherent preparation and substance related tolerance. This is intended to prevent an unscrupulous infringer to design around accurate or absolute values set forth to aid understanding of the present disclosure. The term “step of ˜” used throughout the present disclosure does not mean “step for ˜”.

Throughout the present disclosure, the term “combination thereof” included in expression of a Makushi form means a mixture or combination of at least one selected from the group consisting of elements as recited in the expression of the Makushi form.

As used herein, the term “and/or” includes any and all combinations of one or more of the associated listed items. Expression such as “at least one of” when preceding a list of elements may modify the entire list of elements and may not modify the individual elements of the list.

Throughout the present disclosure specification, the term “biodegradable” means that “a polymer may be chemically broken down in the body to form non-toxic compounds.” In this connection, the decomposition rate is the same as or different from the drug release rate. With the use of biodegradable polymers, the carrier has the property of interacting with the human body without undesirable subsequent effects.

Hereinafter, a lung disease drug delivery carrier according to the present disclosure will be described in detail with reference to an implementation and Example and drawings. However, the present disclosure may not be limited to these implementations and Examples and drawings.

As a technical means for addressing the above technical problem, a first aspect of the present disclosure provides a lung disease drug delivery carrier in which a drug is introduced into a disc particle including polylactide-co-glycolide (PLGA), wherein the carrier delivers and/or releases the drug therein into the lung, wherein the disc particle is 1 μm to 5 μm in size. For example, the disc particle may have a size of 2 μm to 4 μm, but may not be limited thereto.

According to one implementation of the present disclosure, the disc particle may have a size of 3 μm, but may not be limited thereto. The disc particle has a size of 3 μm and has a shape similar to that of red blood cells. Due to its soft nature, the disc particle may significantly reduce the activation of macrophages.

Polylactide-co-glycolide is a biodegradable polymer that is completely decomposed into lactic acid and glycolic acid in the body, but is completely harmless to the human body as the polymer is released as CO₂ out of the body via body metabolism. Thus, the polylactide-co-glycolide is approved by FDA. Further, polylactide-co-glycolide may be formulated in the form of microspheres together with drugs. This formulation prevents the drug from being denatured or aggregated by the external environment such that its activity is changed. Further, polylactide-co-glycolide as a carrier has sustained release property. Thus, at one administration, the drug may last a long time effect in the body. In addition, polylactide-co-glycolide may control the biodegradation period and drug release. That is, varying the copolymer composition and molecular weight thereof may allow controlling the size of the microspheres as a formulation form as needed, and allow the delivery period of the drug to be varied from several weeks to several months. This sustained-release property also has an adjuvant effect, and thus its application range is broad immunologically. However, although polylactide-co-glycolide has been used as a drug carrier in the prior art, it has not been proven that polylactide-co-glycolide is effective in diagnosing or treating lung disease due to intensive accumulation thereof in lung disease sites.

According to one implementation of the present disclosure, the disc particle may further include a polymer selected from the group consisting of polyglycolic acid (PGA), polylactide (PLA), polyglycolide (PG), polyphosphazene, polyiminocarbonate, polyphosphoester, polyanhydride, polyorthoester, and combinations thereof, preferably may further include polyglycolic acid, but may not be limited thereto.

According to one implementation of the present disclosure, the disc particle may be decomposed after 24 hours, but may not be limited thereto. The disc particle is biodegradable using a biodegradable polymer, that is, polylactide-co-glycolide, and thus has the advantage of being harmless to the human body.

According to one implementation of the present disclosure, the drug may include a drug selected from the group consisting of therapeutic agents, contrast agents, diagnostic agents, and combinations thereof, but may not be limited thereto.

The lung disease drug delivery carrier may contain the drug introduced into the disc particle. The carrier may deliver and/or release the drug to the lung to diagnose, image, or treat the lung disease.

According to one implementation of the present disclosure, the therapeutic agent may include one selected from the group consisting of chemotherapeutic compounds, anti-inflammatory agents, anticancer agents, and combinations thereof, but may not be limited thereto.

According to one implementation of the present disclosure, the therapeutic agents may include, but may not be limited to, those selected from the group consisting of cytotoxic agents, cell arresters, alkylating agents, metabolic antagonists, anti-tumor antibiotics, DNA polymerase inhibitors, DNA gyrase inhibitors, topoisomerase inhibitors, mitosis inhibitors, corticosteroids, intercalating agents, antibodies, hormones, antagonists, and combinations thereof.

According to one implementation of the present disclosure, the chemotherapeutic compounds may include those selected from the group consisting of doxorubicin, vinblastine, vincristine, fludarabine, carmustine, asparaginase, fluorouracil, methotrexate, cyclophosphamide, carboplatin, bleomycin, daunorubicin, lomustine, irinotecan, paclitaxel, docetaxel, etoposide, gemcitabine, imatinib, flutamide, hydroxyurea, trastuzumab, curcumin, temozolomide, and combinations thereof, preferably the chemotherapeutic compounds may include doxorubicin, but may not be limited thereto.

According to one implementation of the present disclosure, the drug may include an isotope for nuclear imaging or radiotherapy, but may not be limited thereto.

According to one implementation of the present disclosure, the isotope may include one selected from the group consisting of ⁸⁹Zr, ⁶⁴Cu, ⁶⁸Ga, ⁹⁰Y, ¹⁷⁷Lu, and combinations thereof, but may not be limited thereto.

According to one implementation of the present disclosure, the nuclear imaging may include positron emission tomography (PET), but may not be limited thereto. The positron emission tomography is one of nuclear medicine testing methods capable of displaying physiological, chemical, and functional images of the human body in three dimensions using radiopharmaceuticals that emit positrons. Currently, PET is mainly used to diagnose various cancers, and is known as a useful test for differential diagnosis, staging, recurrence evaluation, and treatment effect determination for cancer. In addition, positron emission tomography may be used to obtain receptor images or metabolic images for heart disease, brain disease and brain function evaluation.

According to one implementation of the present disclosure, the contrast agent may include one selected from the group consisting of USPIO, SPIO, Gd chelate, magnetic nanoparticles, and combinations thereof, but may not be limited thereto.

According to one implementation of the present disclosure, the contrast agent may include an optical activator, but may not be limited thereto.

According to one embodiment of the present disclosure, the optical activator may include a dye selected from the group consisting of fluorescent dyes, cyanine, coumarin, anthracene, acridine, Texas red, fluorescein isothiocyanate (FITC), and combinations thereof, but is not limited thereto.

According to one embodiment of the present disclosure, the optical activator may include a chromophore including a fluorescent chromophore, but is not limited thereto.

According to one embodiment of the present disclosure, the optical activator may include a fluorescent molecule selected from the group consisting of a green fluorescent protein, a fluorescent chromophore, fluorescein isothiocyanate (FITC), and combinations thereof, but is not limited thereto.

The green fluorescent protein refers to a protein capable of emitting green light in a living body to observe how proteins function in a living body. A gene of a fluorescent protein may be attached to a protein to be tracked and then may be injected into a cell. Thus, the movement, location, and growth process of the protein may be easily identified based on the green fluorescent protein. Green fluorescent protein enables observation of phenomena occurring in the human body that otherwise could not be observed with the eye before, thus tracking the proliferation of neurons, the spread of cancer cells, or the destruction of brain neurons in Alzheimer's patients.

According to one embodiment of the present disclosure, as the molar ratio of lactic acid in PLGA of the disc particle increases, the decomposition rate and drug release rate of the lung disease drug delivery carrier may be delayed, but is not limited thereto.

According to one embodiment of the present disclosure, the lung disease may include one selected from the group consisting of pulmonary fibrosis, lung cancer, asthma, and combinations thereof, but is not limited thereto.

First, when the lung disease is pulmonary fibrosis, the lung disease drug delivery carrier may include nintedanib as a drug, but is not limited thereto.

According to one embodiment of the present disclosure, the nintedanib may reduce the consolidation region of the lung, but is not limited thereto.

The pulmonary fibrosis according to the present disclosure may mean idiopathic pulmonary fibrosis, but is not limited thereto. The idiopathic pulmonary fibrosis is a disease having an unknown cause and is known to have an average survival period of less than 3 years after diagnosis. To date, there is no therapy for complete recovery of idiopathic pulmonary fibrosis other than lung transplantation. Several drugs, including nintedanib and pirfenidone, are used as inhibitors to treat the pulmonary fibrosis or prevent worsening of symptoms, but these drugs have low solubility and low bioavailability of 4.7%, and cause side effects in the body such as hemorrhage, arterial thrombosis, myocardial infarction, and liver toxicity. Accordingly, periodic monitoring and oral administration are needed. In order to increase the effect compared to oral administration while reducing these side effects, nintedanib was loaded into the drug delivery carrier according to the present disclosure to be applied to a pulmonary fibrosis animal model.

Compared to oral administration of nintedanib, when nintedanib is loaded into the lung disease drug delivery carrier according to the present disclosure and delivered through blood vessels, the drug may be continuously released for a longer period of time, and since a large amount thereof is accumulated in the lung, it may be seen that the drug delivery rate is improved and the consolidation region in the lung is clearly reduced. In addition, side effects may be reduced, and the lung volume may be recovered compared to simple oral administration.

Meanwhile, when the lung disease is lung cancer, the drug may include doxorubicin.

The doxorubicin itself may be directly administered into blood vessels, but is known to cause various side effects, and the most serious thereof is cardiac toxicity. In order to eliminate or minimize the side effects of doxorubicin, a drug delivery carrier containing doxorubicin and doxorubicin alone were compared.

When the doxorubicin-loaded drug delivery carrier is injected into the lung, the location where cancer cells are metastasized and the location of the drug released from the drug delivery carrier are similar to each other, whereas when the doxorubicin is injected alone into the human body, there may be a problem in that doxorubicin is also disposed in major organs. That is, since the drug delivery carrier allows doxorubicin to be delivered intensively to the lung, when doxorubicin is administered alone for a long period of time, cardiac toxicity appears, but in the doxorubicin-loaded drug delivery carrier, the cardiac toxicity was not observed.

Meanwhile, when the lung disease is asthma, the drug may include curcumin. The curcumin refers to a component that has anti-inflammatory properties and is regarded as a potential anti-asthmatic agent. In this regard, there is a problem that the curcumin is unstable and has low bioavailability, and is excreted through the liver and kidney even when administered orally, and has low bioavailability even when administered intravenously. However, when the drug delivery carrier is intravenously administered after loading the curcumin into the lung disease drug delivery carrier according to the present disclosure, the low bioavailability of curcumin may be overcome.

Meanwhile, the lung disease drug delivery carrier according to one embodiment of the present disclosure may be injected into the human body orally or by injection, but is not limited thereto. For example, the lung disease drug delivery carrier may be prepared in formulations of syrups, tablets, or capsules to be administered orally, or may be administered into the human body by intravenous injection.

For example, the drug nintedanib was administered orally to patients in a tablet form, but when the lung disease drug delivery carrier according to the present disclosure includes nintedanib, the drug delivery carrier may be injected through the vein (blood vessels).

In addition, for example, the doxorubicin, or the lung disease drug delivery carrier loaded with doxorubicin according to the present disclosure may be administered intravenously.

In general, when the drug delivery carrier is administered intravenously into the body, a distribution pattern in the body may vary greatly depending on the size and shape of the drug delivery carrier. For example, a drug delivery carrier with a diameter of 7 μm or more may be mechanically filtered by the capillary of the lung, but a drug delivery carrier with a diameter of 2 μm to 5 μm is accumulated in the reticuloendothelial system (RES) and the like of the lung, liver or spleen. That is, when a drug delivery carrier with a diameter of 2 μm or more is accumulated in the RES and the like of the lung, liver, or spleen for a long time, side effects such as pulmonary embolism and pulmonary fibrosis may occur. Meanwhile, drug delivery carriers with a diameter of several hundred nm are mainly accumulated in the liver and spleen, not in the lung, and particles with a diameter of less than 20 nm may be quickly eliminated through the kidney.

Meanwhile, a change in shape of the drug delivery carrier may also indicate a change in vivo distribution. Illustratively, about 4-fold more a disc-shaped drug delivery carrier than a spherical drug delivery carrier is accumulated. The degree of accumulation of the drug delivery carriers may be confirmed by labeling and observing radioactive isotopes on the surfaces of the spherical drug delivery carrier and the disc-shaped drug delivery carrier.

Hereinafter, the present disclosure will be described in more detail based on Examples, but the following Examples is for illustrative purposes only and is not intended to limit the scope of the present disclosure.

### [Example 1] Preparation of Disc Particle

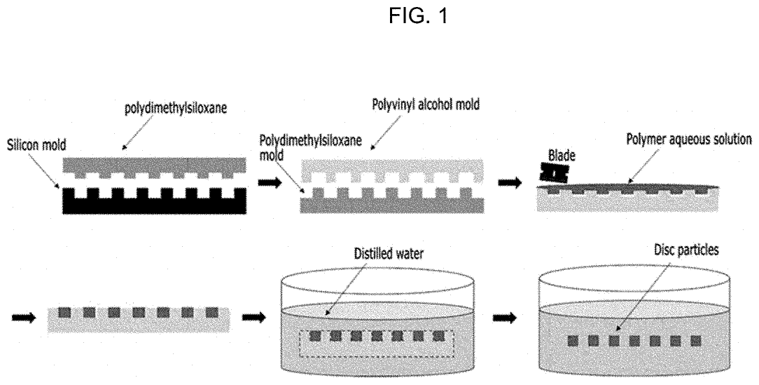

Referring to FIGS. 1 to 3, the method for preparing the disc particle will be described.

FIG. 1 is a schematic diagram of the disc particle preparation process according to one Example of the present disclosure.

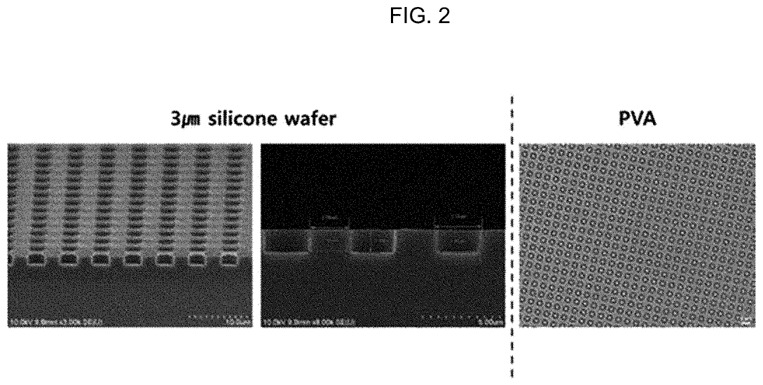

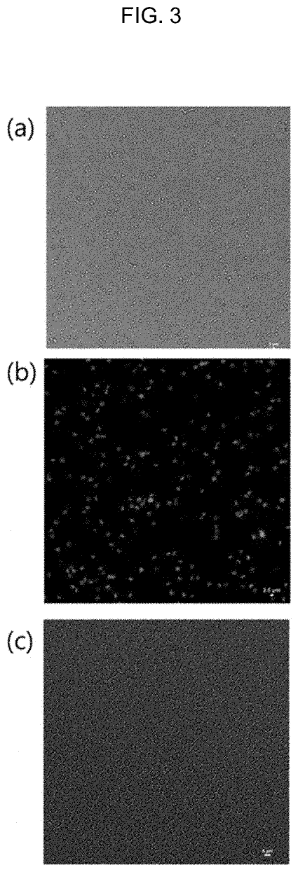

First, a silicon mold having millions of pillars having a width of 3 μm and a depth of 1.5 μm was produced using electron beam lithography. FIG. 2 is a scanning electron microscope image of a silicon mold according to one Example of the present disclosure. Referring to FIG. 2, it may be seen that the silicon mold having millions of pillars having a width of 3 μm and a depth of 1.5 μm is formed. Thereafter, a polydimethylsiloxane layer was deposited on the silicon mold to prepare a polydimethylsiloxane mold having pillars having the same size and shape as that of the silicon mold. Subsequently, a polyvinyl alcohol layer was deposited on the polydimethylsiloxane mold to prepare a polyvinyl alcohol mold having the same pillars as those of the polydimethylsiloxane mold.

A polymer aqueous solution containing polylactide-co-glycolide was deposited on the polyvinyl alcohol mold and then subjected to polymerization via exposure to UV light. Then, the polyvinyl alcohol mold was dissolved in distilled water, and disc particles having a width of 3 μm were collected via centrifugation. (a) OF FIG. 3 is a scanning electron microscope image of a disc particle with a width of 1 μm; (b) OF FIG. 3 is a scanning electron microscope image of a disc particle containing cyanine according to one Example of the present disclosure; (c) OF FIG. 3 is a scanning electron microscope image of a 5 μm width disc particle. Referring to (b) OF FIG. 3, it may be identified that the disc particle according to Example 1 has a shape similar to that of red blood cells.

### [Example 2] Preparation of Lung Disease Drug Delivery Carrier

A polymer aqueous solution containing polylactide-co-glycolide and doxorubicin was deposited on the polyvinyl alcohol mold prepared in Example 1 and then subjected to polymerization via exposure to UV light. Subsequently, the polyvinyl alcohol mold was dissolved in distilled water, and a lung disease drug delivery carrier having a width of 3 μm was collected via centrifugation.

### Comparative Example 1

Mesitylene was added to an aqueous solution of CTAB and NaOH. TEOS was added thereto while stirring at 80° C. Then, the mixture was washed with methanol and dried at 65° C. for one day. Crude MSN was added to methanol and HCl aqueous solution, and sonication was performed. Then, CTAB and mesitylene were removed therefrom while stirring at 50° C. MSN was washed with methanol and dried at 65° C. The dried MSN along with APTES was added to a toluene solution and reaction occurred at 110° C. for 15 hours. Thereafter, MSN-NH2 was washed with ethanol and hexane and then dried at 65° C. for one day. MSN binds to hyaluronic acid via peptide bonds. MSN binding to hyaluronic acid was added to D.I water and then doxorubicin was added thereto while stirring. In this way, mesoporous silica nanoparticles having a self-assembled particle structure were prepared.

### Comparative Example 2

In Comparative Example 2, only the drug was delivered without a separate disc particle. The drug employed the same doxorubicin as in Example 2.

### Experimental Example

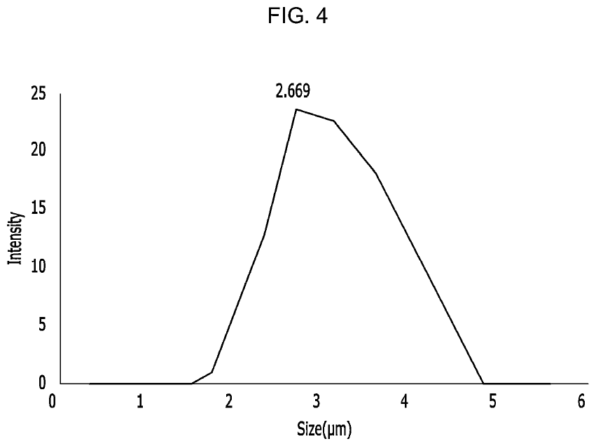

FIG. 4 is a graph of the size distribution of disc particles according to one example of the present disclosure.

Referring to FIG. 4, the disc particle according to one Example of the present disclosure has an average diameter of 2.669 μm.

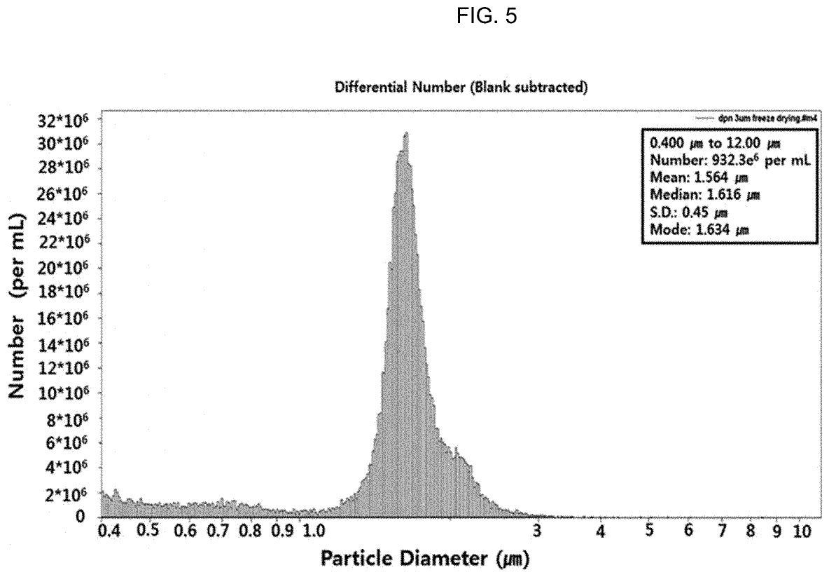

FIG. 5 is a graph of the distribution of the number of disc particles according to one example of the present disclosure.

Referring to FIG. 5, it may be seen that 486.1 e⁶ disc particles are produced per one polyvinyl alcohol mold.

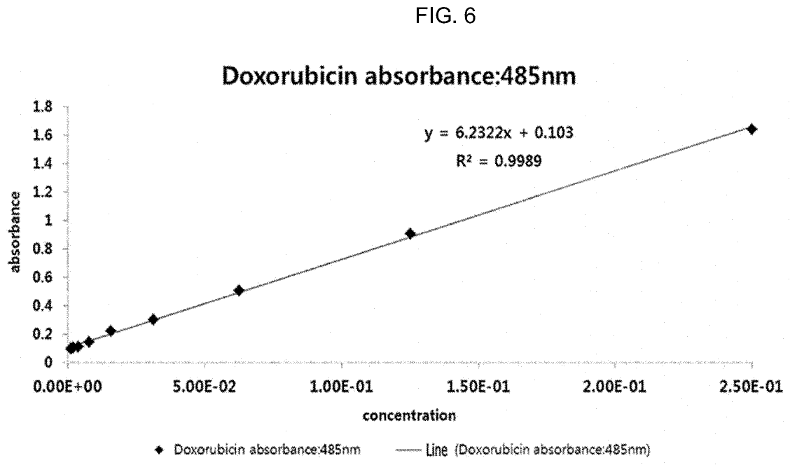

FIG. 6 is a graph of the absorbance of a drug of a lung disease drug delivery carrier according to one example of the present disclosure.

Referring to FIG. 6, the drug loading amount of the lung disease drug delivery carrier according to Example 2 may be identified. Thus, it may be identified that the absorbance of doxorubicin is proportional to the concentration of doxorubicin.

(a) OF FIG. 7 is a table of drug loading amounts of lung disease drug delivery carriers according to one Example of the present disclosure and Comparative Example; (b) OF FIG. 7 is a graph of the drug release amount of the lung disease drug delivery carrier according to one Example of the present disclosure. The drug loading amount may be expressed as (weight of drug contained in disc particle/weight of disc particle)×100.

Referring to (a) OF FIG. 7, it may be identified that the drug loading amount of the drug carrier according to Comparative Example 1 is 9.18%, whereas the drug loading amount of the lung disease drug delivery carrier according to Example 2 is 52.56%, which is about 6 times of the drug loading amount of the drug carrier according to Comparative Example 1.

Referring to (b) OF FIG. 7, it may be identified that the lung disease drug delivery carrier according to Example 2 of the present disclosure releases the drug quickly while initially staying in the lungs for 8 hours and thereafter, releases the drug constantly regardless of time.

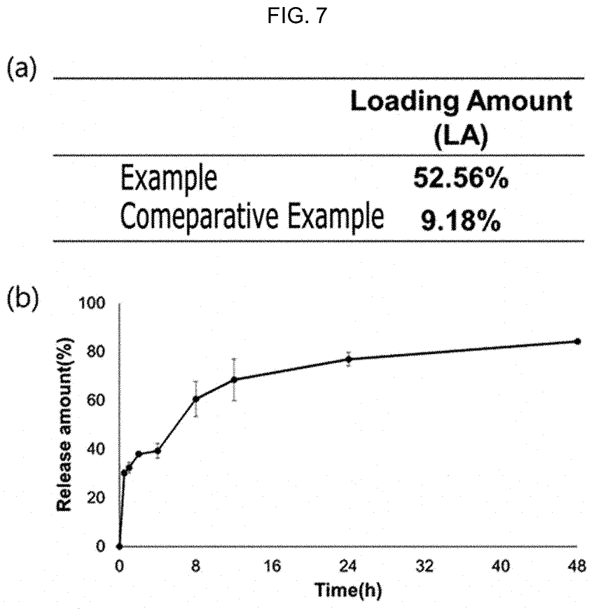

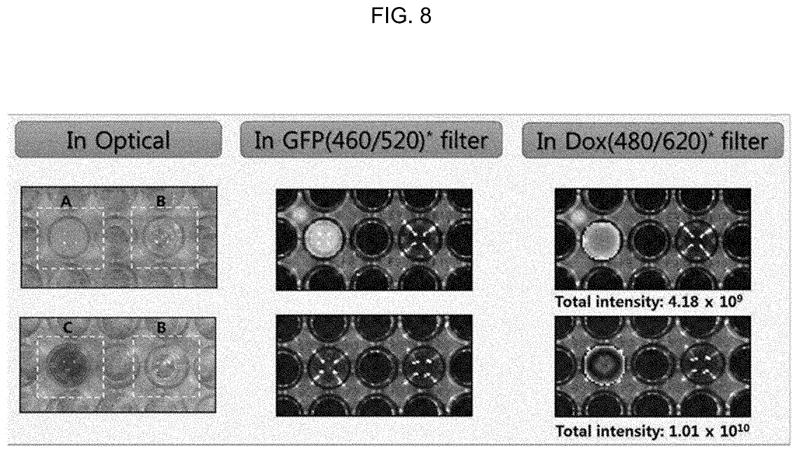

FIG. 8 is an optical microscope image of the lung disease drug delivery carrier and doxorubicin according to one Example of the present disclosure. A is an optical microscope image of a lung disease drug delivery carrier according to Example 2 of the present disclosure as diluted in phosphate buffered physiological saline. B is an optical microscope image containing only phosphate buffered physiological saline. C is an optical microscope image of doxorubicin diluted in phosphate buffered physiological saline. It may be identified that both the disc particle and the lung disease drug delivery carrier exhibit fluorescence only in a Dox filter.

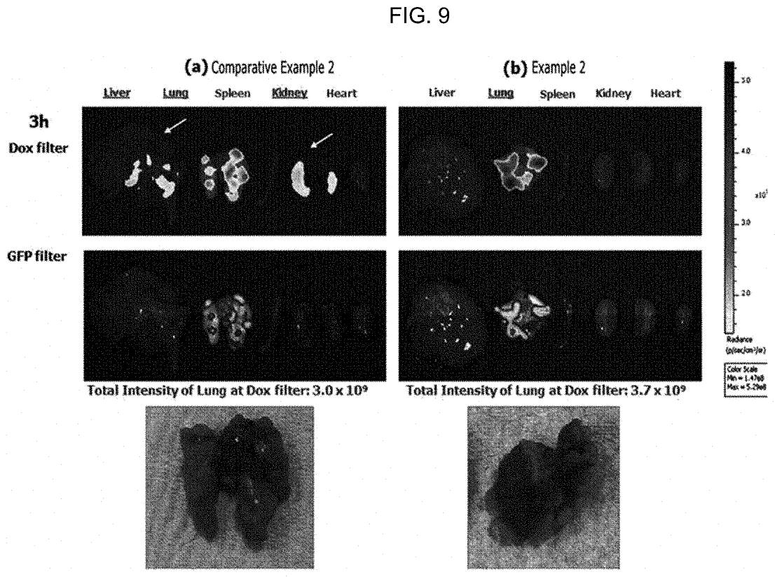

FIG. 9 is an optical microscope image of major organs 3 hours after injecting a lung disease drug delivery carrier according to one Example of the present disclosure and Comparative Example into a lung cancer metastasis model. A GFP filter image may identify the presence or absence of cancer and a location thereof by labeling cancer cells with GFP (green fluorescent protein).

Referring to FIG. 9, it may be identified that the drug according to Comparative Example 2 is accumulated in the liver and kidneys in addition to the lungs of the lung cancer metastasis model, whereas the lung disease drug delivery carrier according to Example 2 is concentrated only in the lung cancer site of the lung cancer metastasis model.

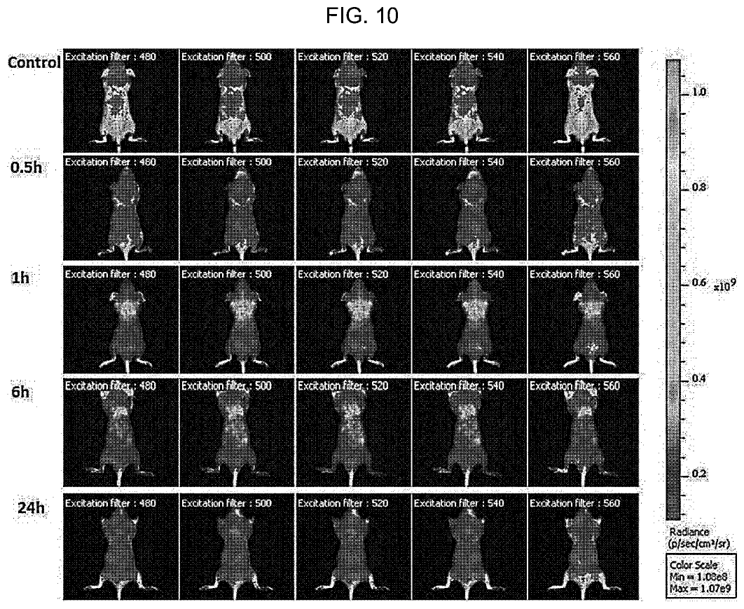

FIG. 10 is an optical microscope image of a lung cancer metastasis model injected with a lung disease drug delivery carrier according to one Example of the present disclosure.

Referring to FIG. 10, it may be identified that lung disease drug delivery carriers according to Example 2 of the present disclosure accumulate in the lungs, then gradually biodegrade, such that most thereof is removed after 6 hours.

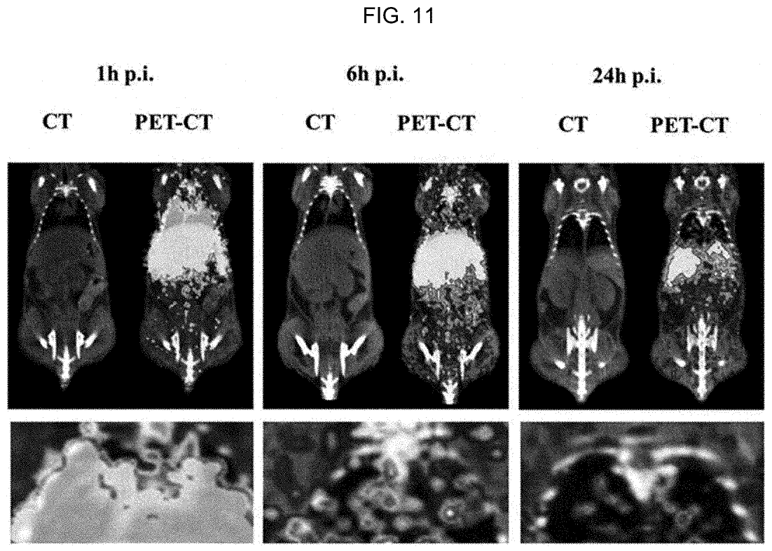

FIG. 11 is a computed tomography and positron emission tomography image of a lung cancer metastasis model injected with a lung disease drug delivery carrier according to one Example of the present disclosure.

Referring to FIG. 11, it may be identified that lung disease drug delivery carriers according to Example 2 of the present disclosure accumulate in the lungs, gradually biodegrade, such that a large amount thereof is removed after 6 hours.

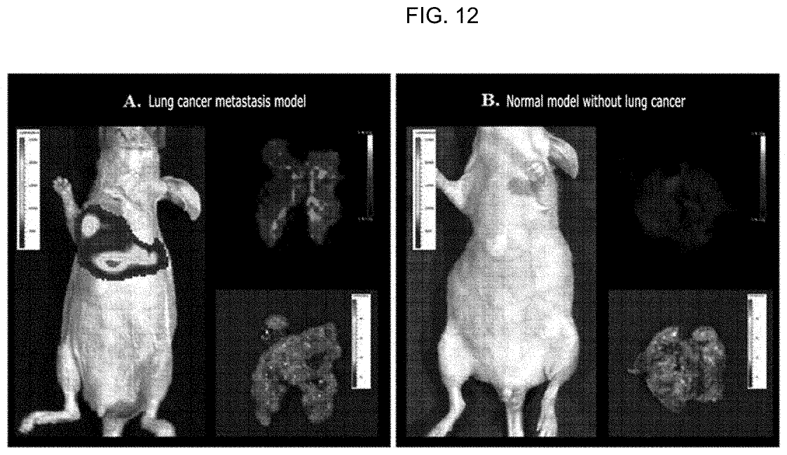

FIG. 12 is a positron emission tomography image of a lung cancer metastasis model and a normal model injected with a lung disease drug delivery carrier according to one Example of the present disclosure.

Referring to FIG. 12, it may be seen that the lung disease drug delivery carrier according to Example 2 of the present disclosure accumulates in a larger amount in the lung cancer metastasis model, compared to the normal model without lung cancer. Thus, using the lung disease drug delivery carrier according to Example 2, lung diseases such as lung cancer may be intensively diagnosed, and may be treated by effectively delivering the drug thereto.

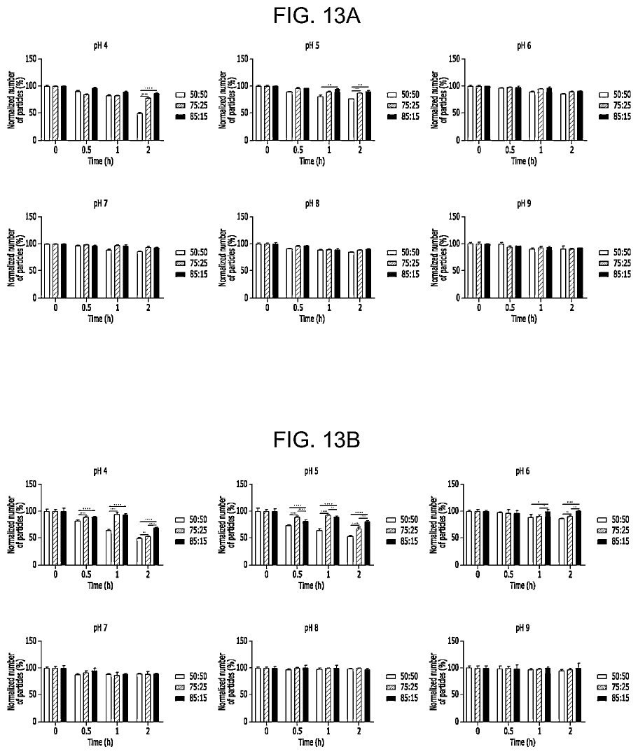

FIG. 13A is a graph showing the degree of decomposition according to a molar ratio of lactic acid in PLGA disc particles according to one embodiment of the present disclosure, and FIG. 13B is a graph showing the degree of decomposition according to a molar ratio of lactic acid in drug-loaded PLGA disc particles according to one embodiment of the present disclosure. Specifically, in 50:50, 75:25, and 85:15, the first elements 50, 75, and 85 refer to the molar ratios of lactic acid in PLGA, and the second elements 50, 25, and 15 refer to the molar ratios of glycolic acid in PLGA, and the drug may mean nintedanib.

Referring to FIGS. 13A and 13B, the decomposition rates of the PLGA disc particles and the drug-loaded PLGA disc particles may be delayed as the ratio of lactic acid increases regardless of pH.

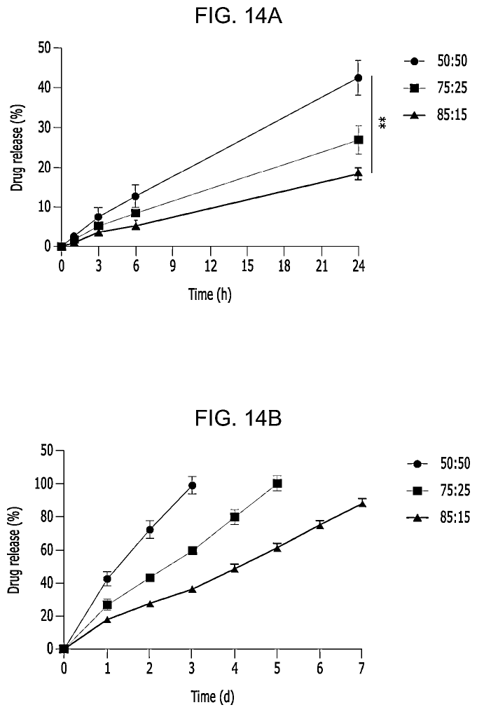

FIGS. 14A and 14B are graphs showing the degree of release of nintedanib over time from PLGA disc particles loaded with nintedanib according to one embodiment of the present disclosure. Referring to FIGS. 14A and 14B, it may be confirmed that the higher the ratio of lactic acid in PLGA, the slower the release rate of the drug, thereby continuously releasing the drug.

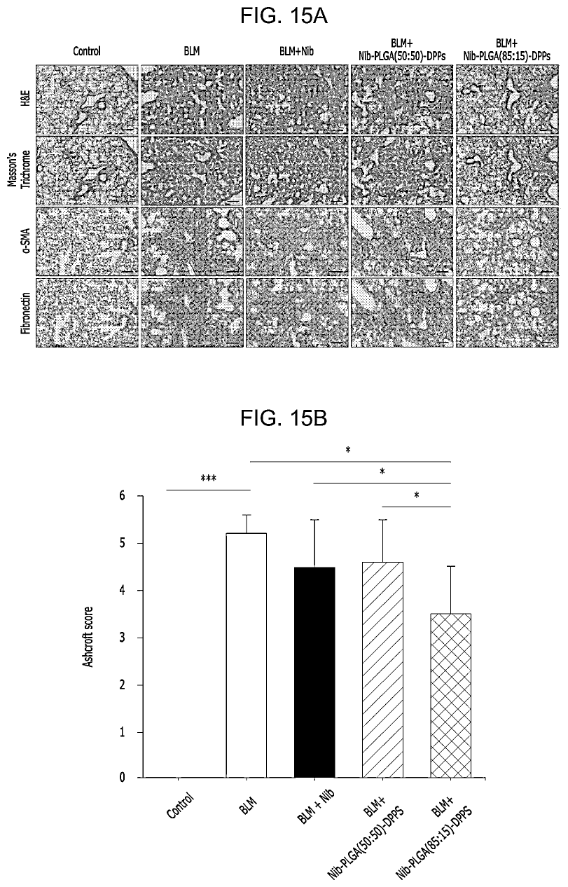

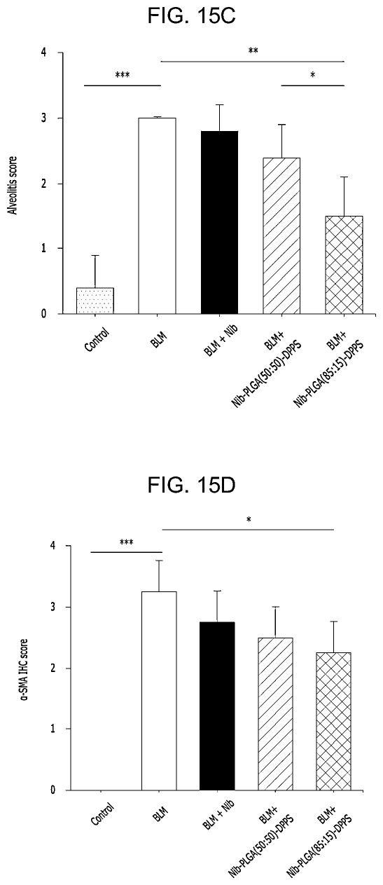

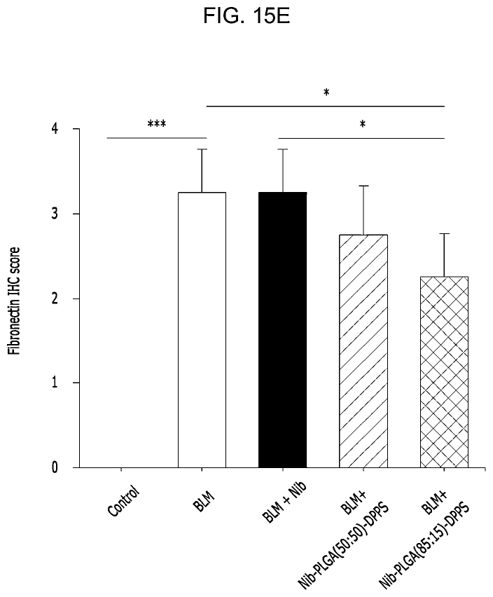

FIG. 15A illustrates an effect of nintedanib-loaded PLGA disc particles according to one embodiment of the present disclosure, and FIGS. 15B to 15E illustrate experimental results according to each comparison group. Specifically, the experiments in FIGS. 15A to 15E are performed by a control, BLM administered with only bleomycin (BLM), BLM+Nib orally administered with nintedanib (Nib) and BLM, BLM+Nib-PLGA(50:50)-DPPs administered with BLM and PLGA disc particles loaded with Nib and with a molar ratio of lactic acid to glycolic acid of 50:50, and BLM+Nib-PLGA(85:15)-DPPs administered with BLM and PLGA disc particles loaded with Nib and with a molar ratio of lactic acid to glycolic acid of 85:15. FIGS. 15A to 15E illustrate results obtained by evaluating tissue morphology and degrees of inflammation and fibrosis through histopathological and immunohistochemical changes. Also, a scale bar in FIG. 15A is 100 μm.

Referring to FIGS. 15A to 15E, in a model administered with BLM and BLM+Nib, an inflammatory response is shown and the formation of a dense fibrous mass is shown, whereas it may be confirmed that the fibrous mass and the inflammatory response are reduced when the drug delivery carrier BLM+Nib-PLGA-DPPs was used. In addition, as a result of comparing the severity of pulmonary fibrosis with Ashcroft scores in FIG. 15B, it may be seen that as compared to BLM or BLM+Nib, a significantly low value is shown when using a drug delivery carrier loaded with Nib, and as the ratio of lactic acid in PLGA DPPs is increased, the bioavailability of the drug is improved and an improved therapeutic effect may be exhibited. In addition, FIG. 15C illustrates results of inflammation scores, and it may be seen that in a BLM+Nin-PLGA (85:15) group, the infiltration amount of inflammatory cells and the thickness of the alveolar septum are decreased, and the inflammation score was significantly low, as compared to the BLM and BLM+Nib groups.

In addition, FIGS. 15D and 15E illustrate indicators for evaluating the degree of pulmonary fibrosis when drugs are administered, and in the BLM+Nin-PLGA (85:15) treated group, the expression levels of α-SMA and fibronectin were significantly reduced as compared to other groups, which means that fibrosis was effectively inhibited.

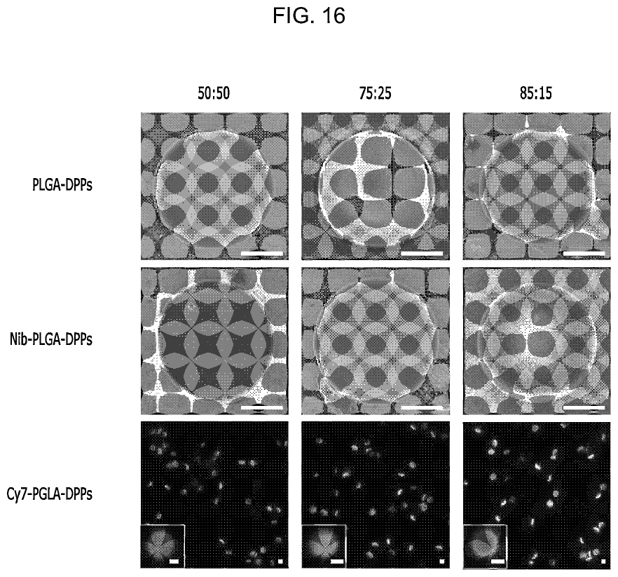

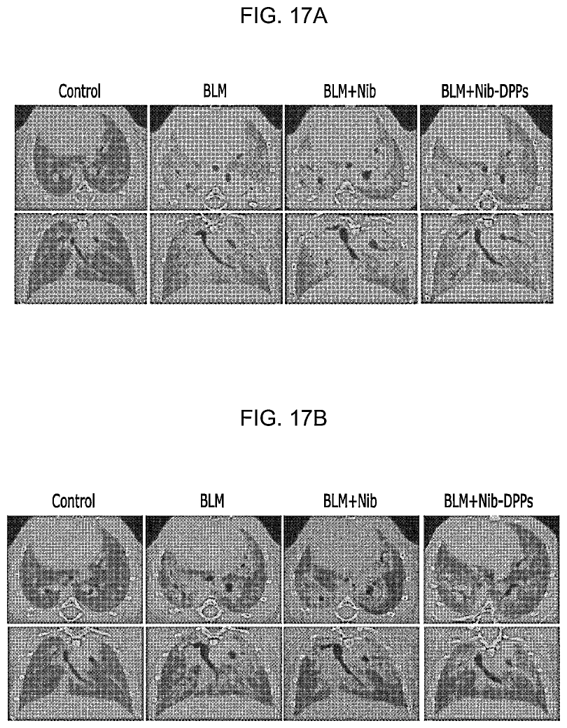

FIG. 16 is an SEM image expressing the shape of PLGA disc particles according to one embodiment of the present disclosure, and the indicated scale bar means 1 μm. In addition, FIGS. 17A and 17B are micro-CT images of a control group, a BLM administered group, a BLM and Nib administered group, and a BLM and Nib-DPPs administered group. At this time, Nib is orally administered in the BLM and Nib administered group.

Referring to FIGS. 16 to 17B, it may be seen that the lung in which pulmonary fibrosis was induced by accumulation of BLM in the lung had a significantly reduced volume compared to the control group (lungs in which pulmonary fibrosis was not induced), and the lung volume was clearly recovered when using a drug delivery carrier (Nib-DPPs) compared to when administering only nintedanib. That is, in the case of using the lung disease drug delivery carrier containing PLGA disc particles according to the present disclosure, it may be seen that the bioavailability of nintedanib may be improved to reduce side effects and be effectively applied to the treatment of idiopathic pulmonary fibrosis.

Meanwhile, referring to FIG. 16, hydrodynamic diameters, zeta potentials, and the like of PLGA-DPP and Nib-PLGA-DPP may be measured. The hydrodynamic diameter and the zeta potential may be measured through dynamic light scattering technique. Referring to Table 1 below, the average particle sizes of PLGA(50:50)-DPPs, PLGA(75:25)-DPPs, and PLGA(85:15)-DPPs were 2.82±0.47 μm, 2.84±0.65 μm, and 2.84±0.82 μm, respectively, and the ratio of lactic acid to glycolic acid in PLGA did not affect the particle size. In addition, the zeta potential value of the PLGA (50:50)-DPP was measured as about −30 mV, which was similar to those of PLGA (75:25)-DPP and PLGA (85:15)-DPP.

Meanwhile, the particle size of PLGA-DPPs loaded with nintedanib was measured as about 2.87 μm, which means that the loading of nintedanib did not significantly affect the particle size of PLGA-DPPs. In addition, the zeta potential for each lactide:glycolide ratio of Nib-PLGA-DPP was similarly measured. This is because particles having a zeta potential value greater than +30 mV or smaller than −30 mV are aggregated or not due to van der Waals forces, that is, Nib-PLGA-DPP is stable.

TABLE 1

| | Particle | Zeta | Loading | |
|---|---|---|---|---|
| | size ± | potential ± | amount ± | |
| | SD (μm) | SD (mV) | SD (%) | PDI |
| PLGA(50:50)-DPP | 2.82 ± 0.47 | −30.9 ± 4.52 | — | 0.193 |
| PLGA(75:25)-DPP | 2.84 ± 0.65 | −30.4 ± 3.89 | — | 0.170 |
| PLGA(85:15)-DPP | 2.82 ± 0.82 | −31.9 ± 3.95 | — | 0.128 |
| Nib-PLGA(50:50)- | 2.86 ± 0.19 | −23.0 ± 3.81 | 15.82 ± 0.12 | 0.162 |
| DPP | | | | |
| Nib-PLGA(75:25)- | 2.87 ± 0.14 | −23.4 ± 3.99 | 15.54 ± 0.09 | 0.184 |
| DPP | | | | |
| Nib-PLGA(85:15)- | 2.89 ± 0.29 | −24.9 ± 5.42 | 15.71 ± 0.12 | 0.158 |
| DPP | | | | |

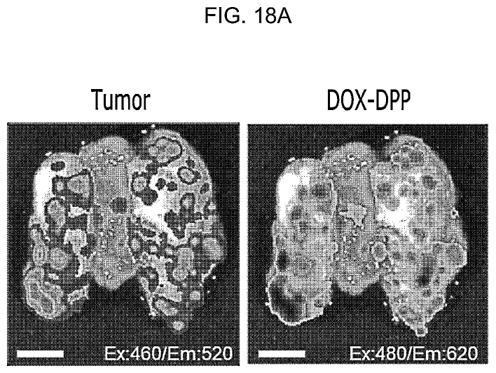

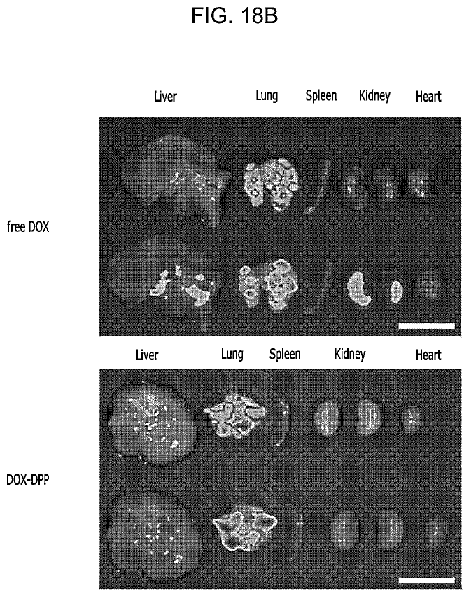

FIG. 18A is an image of photographing the locations of cancer tumor and the locations of an injected DOX-DPP (doxorubicin-loaded disc-shaped drug delivery carrier) in an animal model, and FIG. 18B illustrates comparison of the locations of cancer tumor in major organs and amounts of doxorubicin alone (free DOX) or DOX-DPP in major organs. In this regard, the scale bar in FIG. 18A means 5 mm, and the scale bar in FIG. 18B means 20 mm. In addition, the cancer is a cancer cell expressing a GFP green fluorescent protein.

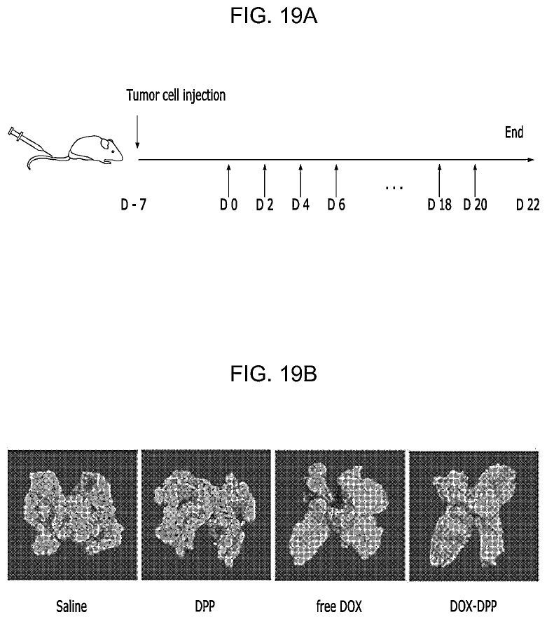

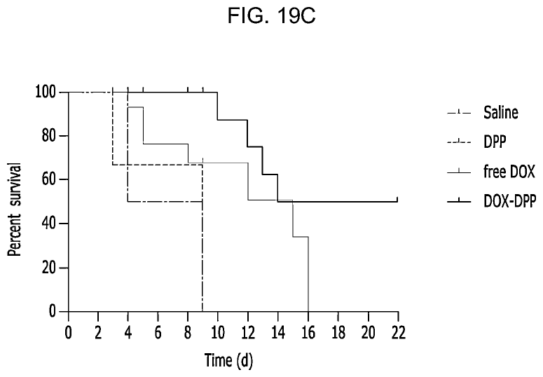

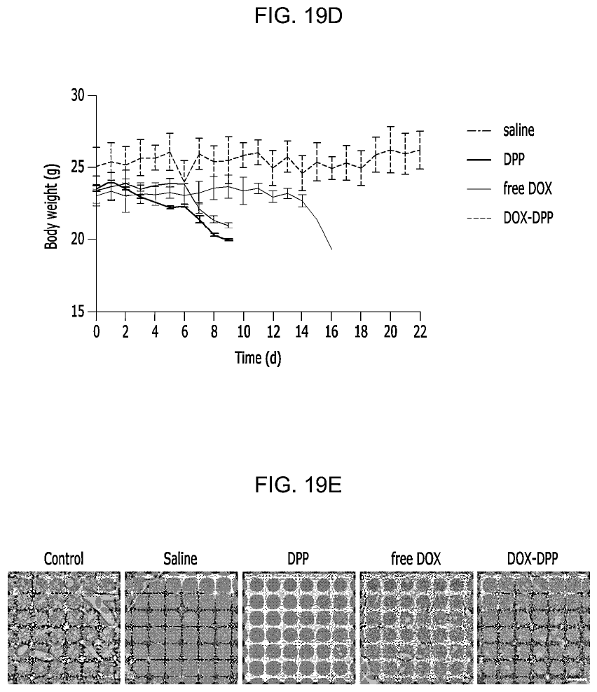

FIG. 19A is a schematic diagram expressing a treatment time after injecting cancer cells into an animal (rat), and FIG. 19B illustrates lung specimens of C57BL/6 mice with lung cancer, which are treated with saline, a drug delivery carrier (DPP), doxorubicin (free DOX), and a doxorubicin-loaded drug delivery carrier (DOX-DPP). In addition, FIG. 19C is a graph showing a change in survival rate of rats according to treated drugs over time, FIG. 19D is a graph showing a change in body weight of the rat according to treated drugs over time, and FIG. 19E illustrates lung tissues of the rat according to treated drugs. In this regard, the scale bar in FIG. 19E means 20 μm.

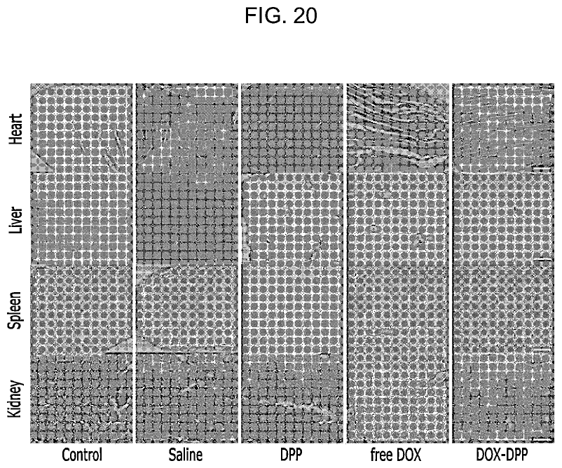

FIG. 20 illustrates tissue images of each organ of a rat according to treated drugs.

Referring to FIGS. 18A to 20, it may be seen that the location where the cancer cells were metastasized and the location where the drug (DOX-DPP) was delivered are very similar to each other. However, it may be seen that when only doxorubicin is administered (free DOX), the drug is distributed even in major organs, whereas when the doxorubicin-loaded drug delivery carrier (DOX-DPP) is used, the drug is delivered preferentially to the lung, which means that the side effects of an anticancer drug (doxorubicin) in the body may be reduced through the drug delivery carrier.

In addition, it may be seen that among rats injected with cancer cells into the lungs, more metastatic tumor nodules appeared in an anticancer drug-alone delivered group (free DOX), and tumor nodules were decreased using the drug delivery carrier (DOX-DPP). In addition, as a result of histological analysis, when doxorubicin was administered using a drug delivery carrier, symptoms of narrowing of the alveolar space due to inflammatory responses or cancer cells were more recovered than when doxorubicin was administered alone. In addition, it may be confirmed that as compared to a case of administering only doxorubicin, a change in body weight due to the toxicity of the anticancer agent is small in the group applied with the doxorubicin-loaded drug delivery carrier.

In addition, doxorubicin is known to have strong cardiotoxicity, and in the case of Free DOX, it may be confirmed that strong inflammatory responses occur in the heart and kidney. However, in the case of using the drug delivery carrier (DOX-DPP), the inflammation was not confirmed in the heart and kidney.

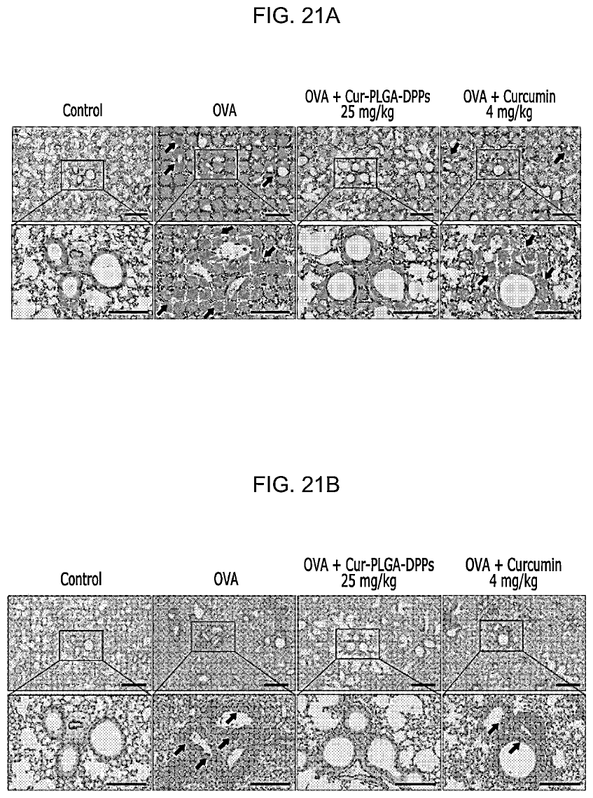

FIGS. 21A and 21B are lung tissue images of rats with asthma according to treated drugs. Specifically, FIG. 21A is an image of H&E staining, FIG. 21B is an image of PAS staining, and rats of FIGS. 21A and 21B mean rats in a control, an ovalbumin administered group (OVA), a group administered with a drug delivery carrier (Cur-PLGA-DPPs) loaded with ovalbumin and curcumin at a concentration of 25 mg/kg (OVA+Cur-PLGA-DPPs), and a group administered with ovalbumin and curcumin at a concentration of 5 mg/kg (OVA+Curcumin). In addition, in FIGS. 21A and 21B, the scale bar of the upper part is 400 μm and the scale bar of the lower part is 200 μm.

Referring to FIGS. 21A and 21B, it may be seen that as a result of administering a drug delivery carrier loaded with the same amount of curcumin (OVA+Cur-PLGA-DPPs), the inflammatory cell infiltration in the peribronchial area is significantly reduced compared to administration of only curcumin (OVA+Curcumin). That is, in the case of using a disc-shaped PLGA drug delivery carrier when delivering curcumin, it means that a more improved therapeutic effect may be exhibited even with the same dose.

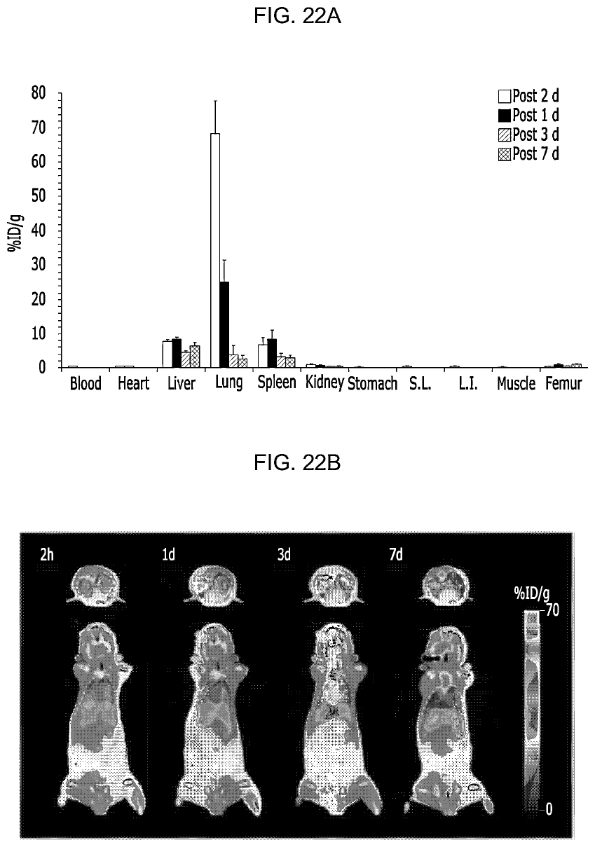

FIG. 22A illustrates an in-vivo distribution of radioactive isotope-labeled disc-shaped drug delivery carriers, and FIG. 22B is an image of photographing rats injected with the disc-shaped drug delivery carrier.

Referring to FIGS. 22A and 22B, it may be seen that about 70% of the injected amount of the disc-shaped drug delivery carrier is delivered to the lung after 2 hours of injection and is decomposed over time.

The description of the present disclosure above is for illustration purposes only. Those of ordinary skill in the technical field to which the present disclosure belongs may understand that it is possible to easily vary the present disclosure into other concrete forms without changing the technical idea or essential characteristics of the present disclosure. Therefore, the examples described above are illustrative in all respects and should be understood as non-limiting. For example, components described in a single form may be implemented in a distributed manner. Likewise, components that are described as being distributed may be implemented in a combined form.

The scope of the present disclosure is indicated by the claims to be described later rather than the detailed description. The meaning and scope of the claims and all changes or modifications derived from the concept of equivalent should be construed as being included in the scope of the present disclosure.

Although the examples of the present disclosure have been described in detail with reference to the accompanying drawings, the present disclosure may not be limited thereto and may be embodied in many different forms without departing from the technical concept of the present disclosure. Therefore, the examples of the present disclosure are provided for illustrative purposes only but not intended to limit the technical spirit of the present disclosure. The scope of the technical spirit of the present disclosure may not be limited thereto. Therefore, it should be understood that the above-described examples are illustrative in all aspects and do not limit the present disclosure. The protective scope of the present disclosure should be construed based on the following claims, and all the technical concepts in the equivalent scope thereof should be construed as falling within the scope of the present disclosure.

What is claimed is:

1. A lung disease drug delivery carrier, wherein the lung disease drug delivery carrier includes a disc particle having a diameter of 2 μm to 4 μm,
the disc particle is injected into the human body,
the disc particle includes a polymer selected from the group consisting of polyglycolic acid (PGA), polylactide (PLA), polyglycolide (PG), polyphosphazene, polyiminocarbonate, polyphosphoester, polyanhydride, polyorthoester, and combinations thereof, polylactide-co-glycolide (PLGA), and a drug,
the disc particle is decomposed after 24 hours after being injected into the human body and delivers or releases the drug into a lung,
the lung disease drug delivery carrier is accumulated in the lung, and
the lung disease includes pulmonary fibrosis.

2. The lung disease drug delivery carrier of claim 1, wherein the disc particle has a size of 3 μm.

3. The lung disease drug delivery carrier of claim 1, wherein the drug includes one selected from a group consisting of a therapeutic agent, a contrast agent, a diagnostic agent, and combinations thereof.

4. The lung disease drug delivery carrier of claim 3, wherein the therapeutic agent includes one selected from a group consisting of a chemotherapeutic compound, an anti-inflammatory agent, an anticancer agent, and combinations thereof.

5. The lung disease drug delivery carrier of claim 4, wherein the therapeutic agent includes one selected from a group consisting of a cytotoxic agent, a cell arrester, an alkylating agent, a metabolic antagonist, an anti-tumor antibiotic, a DNA polymerase inhibitor, a DNA gyrase inhibitor, a topoisomerase inhibitor, a mitosis inhibitor, corticosteroid, an intercalating agent, an antibody, hormone, antagonist, and combinations thereof.

6. The lung disease drug delivery carrier of claim 4, wherein the chemotherapeutic compound includes one selected from a group consisting of nintedanib, doxorubicin, vinblastine, vincristine, fludarabine, carmustine, asparaginase, fluorouracil, methotrexate, cyclophosphamide, carboplatin, bleomycin, daunorubicin, lomustine, irinotecan, paclitaxel, docetaxel, etoposide, gemcitabine, imatinib, flutamide, hydroxyurea, trastuzumab, curcumin, temozolomide, and combinations thereof.

7. The lung disease drug delivery carrier of claim 6, wherein the drug includes nintedanib, wherein the nintedanib reduces a consolidation region of the lung.

8. The lung disease drug delivery carrier of claim 1, wherein the drug includes an isotope for nuclear imaging or radiotherapy.

9. The lung disease drug delivery carrier of claim 8, wherein the isotope includes one selected from a group consisting of ⁸⁹Zr, ⁶⁴Cu, ⁶⁸Ga, ⁹⁰Y, ¹⁷⁷Lu, and combinations thereof.

10. The lung disease drug delivery carrier of claim 8, wherein the nuclear imaging includes positron emission tomography (PET).

11. The lung disease drug delivery carrier of claim 3, wherein the contrast agent includes one selected from a group consisting of USPIO, SPIO, Gd chelate, magnetic nanoparticles, and combinations thereof.

12. The lung disease drug delivery carrier of claim 3, wherein the contrast agent includes an optical activator.

13. The lung disease drug delivery carrier of claim 12, wherein the optical activator includes a dye selected from the group consisting of fluorescent dyes, cyanine, coumarin, anthracene, acridine, Texas red, fluorescein isothiocyanate (FITC), and combinations thereof.

14. The lung disease drug delivery carrier of claim 12, wherein the optical activator includes a chromophore including a fluorescent chromophore.

15. The lung disease drug delivery carrier of claim 12, wherein the optical activator includes a fluorescent molecule selected from the group consisting of a green fluorescent protein, a fluorescent chromophore, fluorescein isothiocyanate (FITC), and combinations thereof.

16. The lung disease drug delivery carrier of claim 1, wherein as a molar ratio of lactic acid in PLGA of the disc particle is increased, the decomposition rate and drug release rate of the lung disease drug delivery carrier are delayed.
